(19) United States
(12) Patent Application Publication
(10) Pub. No.: US 2013/0041227 A1
(43) Pub. Date: Feb. 14, 2013

(54) LIGHTWAND FOR ORAL/NASAL INTUBATION

(71) Applicants: Paul CHAN, Sunnyvale, CA (US); Richard A. JAFFE, Stanford, CA (US)

(72) Inventors: Paul CHAN, Sunnyvale, CA (US); Richard A. JAFFE, Stanford, CA (US)

(21) Appl. No.: 13/564,585

(22) Filed: Aug. 1, 2012

Related U.S. Application Data
(60) Provisional application No. 61/521,580, filed on Aug. 9, 2011.

(51) Int. Cl. A61B 1/06; A61B 1/267

(52) U.S. Cl. 600/199

(57) ABSTRACT

According to some embodiments, a light wand is described that can be used for fast, easy and non-traumatic oral and nasal tracheal intubation. A flexible light wand assembly includes an adapter plate that is compatible with existing standard laryngoscope handles.

### CROSS-REFERENCE TO RELATED APPLICATIONS

This patent application claims the benefit of U.S. Prov. Ser. No. 61/521,580 filed Aug. 9, 2011, which is incorporated by reference herein.

### FIELD

The present disclosure generally relates to a medical device for use in intubation procedures. More particularly, the present disclosure relates generally to a lightwand for use in oral and/or nasal tracheal intubation medical procedures.

### BACKGROUND

Tracheal intubation, commonly simply referred to as intubation, is the placement of a flexible plastic tube into the trachea (windpipe) to maintain an open airway or to serve as a conduit through which to administer certain drugs. It is frequently performed in critically injured, ill or anesthetized patients to facilitate ventilation of the lungs, including medical ventilation, and to prevent the possibility of asphyxiation or airway obstruction.

The most widely used route is orotracheal, in which an endotracheal tube is passed through the mouth and vocal apparatus into the trachea. In nasotracheal procedures, an endotracheal tube is passed through the nose and vocal apparatus into the trachea. Because it is an invasive and extremely uncomfortable medical procedure, intubation is usually performed after administration of general anesthesia and a neuromuscular-blocking drug.

The vast majority of tracheal intubations involve the use of a viewing instrument of one type of another. The conventional laryngoscope consists of a handle containing batteries for powering a light and a set of interchangeable blades made from a material such as stainless steel. The blades are either straight or curved. Direct laryngoscopy is carried usually carried out with the patient lying on his or her back, and the laryngoscope is inserted into the mouth and moved to one side so as to move the tongue out of the line of sight, and depending on the type of blade used, inserted either anterior or posterior to the epiglottis and then lifted with an upwards and forward motion. The move makes a view of the glottis possible.

However, the use of conventional laryngoscopes can be traumatic to the patient, such as by causing damage to the tongue and/or other soft tissues, as well as to the teeth. Additionally, there are a number of situations in which a conventional laryngoscope should not be used. For example, in cases where there are certain types of damage or injury to the mouth; the view is obstructed such as with a traumatized airway; excess tissue impairs the direct view, such as with fatty tissue in an obese patient; and a patient with suspected neck trauma who has an immobilized neck such as with a cervical collar.

An alternative to the use of conventional direct laryngoscopy is the use of a lightwand-type instrument. Lightwand intubation uses a lighted stylet to transilluminate the anterior neck tissues to demonstrate tracheal placement. The lighted stylet is first inserted into an appropriately sized endotracheal tube such that the light is near or at the distal tip of the endotracheal tube. This assembly is then bent into a shape compatible with the patient's anatomy. The endotracheal tube with the lighted tip is then inserted through the patient's mouth. Correct placement of the endotracheal tube is indicated by a specific light pattern such as a distinctive cone-shaped pattern seen through the anterior neck tissues, rather than the diffuse glow that is seen with incorrect placement. The user often performs a jaw-lifting procedure by hand, but since the procedure does not rely on direct line of sight, lightwand intubation does not require a rigid blade or the amount of movement of the neck, cervical spine and other tissues as with conventional direct laryngoscopy. Examples of commercially used lightwand instruments for intubation include: Trachlight™ by Laerdal (now discontinued); Vital Light™ by Vital Signs; Trachlight™ by Rusch; and Surch-Lite™ by Bovie. All of these lightwand instruments are either provided as a self-contained, often single-use unit, containing batteries and lighted stylet, or as a system that includes a lighted stylet and a proprietary lightwand handle.

### SUMMARY

According to some embodiments, a device for use in tracheal (nasal and/or oral) intubation is described. The device includes a base portion including an adapter portion adapted for physical mounting to a laryngoscope handle, such as handles conforming to International Standard ISO 7376; and a flexible elongated member having a distal end and a proximal end, the proximal being fixed to the base portion, and the distal end including a light emitting tip portion capable of emitting light for facilitating airway intubation of a patient when the device is mounted to a laryngoscope handle. According to some embodiments, the device is further adapted to receive DC electric power supplied by the laryngoscope handle for use by a light source. According to some embodiments, the light source is LED-based and according to some other embodiments other types of sources such as based on small bulbs. The device can be un-switched such that light is emitted upon mounting to the DC powered handle (in such embodiments) or can include a switching unit operable by a user to turn on and off light generated by the light source.

According to some embodiments, the device includes a light guide adapted and positioned to guide light received from a laryngoscope handle to the light emitting tip portion. According to some other embodiments, the device includes one or more batteries for supplying power for use in generating light for the light emitting tip portion. In some embodiments the batteries are used to solely power the light source and in other embodiments they are used to supplement or boost power supplied by the laryngoscope handle. According to some embodiments a thumb rest portion is positioned and dimensioned so as to facilitate grasping and manipulation of the device by a user during an intubation procedure on a patient.

According to some embodiments, the light emitting tip portion is adapted to to emit light primarily in a 30 to 60 degree field of view aligned with a longitudinal axis of the distal end of the flexible elongated member, and according to other embodiments, the light emitting tip portion is adapted to also emit light in directions perpendicular to a longitudinal axis of the distal end of the flexible elongated member, so as to aid the user during airway tracheal intubation procedure on a patient by giving the user an indication of the location of the distal end of the device.

According to some embodiments, a method is described of intubating the trachea of a patient. The method including: mounting a device to a laryngoscope handle, the device including a flexible elongated member having a light emitting distal tip portion, and an adapter portion adapted for physical mounting to the laryngoscope handle; inserting the elongated member through an endotracheal tube; and intubating the trachea of the patient by inserting the elongated member and endotracheal tube through the patient's airway. According to some embodiments, the intubation is facilitated by light from the light emitting distal tip portion transilluminating anterior neck tissues thereby demonstrating laryngeal placement. According to some embodiments the method includes bending the elongated member and endotracheal tube to a shape believed to be compatible with the patient's anatomy prior to the insertion. According to some embodiments the type of light emitted includes infrared frequencies such that an infrared viewer can be used to facilitate intubation. According to some embodiments, the laryngoscope handle conforms to International Standard ISO 7376.

### BRIEF DESCRIPTION OF THE DRAWINGS

The inventive body of work will be readily understood by referring to the following detailed description in conjunction with the accompanying drawings, in which:

FIGS. 1A-1C show a general depiction of the light wand, according to some embodiments;

FIG. 1D depicts a standardized laryngoscope handle;

FIGS. 2A-2B illustrate a lighted tip assembly having multi-facetted lens according to some embodiments;

FIGS. 2C-2D illustrate lighted tip assemblies for light wands according to some embodiments;

FIGS. 3A-3G illustrate further details of certain aspects of a light wand device that is mountable to a standard laryngoscope handle, according to some embodiments; and

FIG. 4 is a flow chart showing some aspects of a method of intubating an airway of a patient, according to some embodiments.

### DESCRIPTION

A detailed description of the inventive body of work is provided below. While several embodiments are described, it should be understood that the inventive body of work is not limited to any one embodiment, but instead encompasses numerous alternatives, modifications, and equivalents. In addition, while numerous specific details are set forth in the following description in order to provide a thorough understanding of the inventive body of work, some embodiments can be practiced without some or all of these details. Moreover, for the purpose of clarity, certain technical material that is known in the related art has not been described in detail in order to avoid unnecessarily obscuring the inventive body of work.

According to some embodiments, a novel lightwand for fast, easy and non-traumatic oral and nasal tracheal intubation is described. Many of the embodiments are designed to be widely acceptable and compatible with existing intubation devices and accessories.

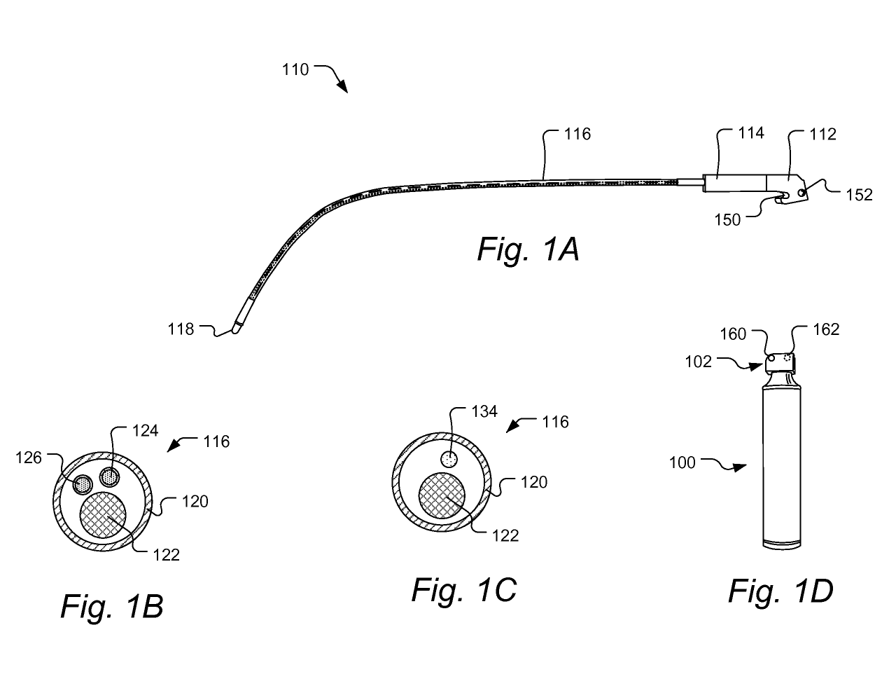

FIGS. 1A-1C show a general depiction of the light wand, according to some embodiments. The lightwand 110 consists of the following key components: adaptor plate 112, base 114, flexible/semi rigid stylet 116, and lighted tip assembly 118.

According to some preferred embodiments, the adapter plate 112 has a hook 150 (also referred to as a hinge slot) and a raised assembly 152 that are designed to match a post and detent on a standardized laryngoscope handle assembly. FIG. 1D depicts a standardized laryngoscope handle assembly 100 having an anchor block 102 (also referred to as a hook-on fitting) that includes a notch that accepts the anchor plate 112, and a post 160 (also referred to as a hinge pin) and detents 162. According to some preferred embodiments, the adapter plate 112 conforms to International Standard ISO 7376, such as described in the publication 7376:2003(E) which is incorporated by reference herein. According to some other embodiments the adapter plate 112 is configured for mounting on another type of laryngoscope handle.

According to some embodiments, adaptor plate 112, can be a different shape and style so as to mount the lightwand to other commonly available laryngoscope handles.

According to some embodiments, the adapter plate can be used for a passive, physical connection only. According to some preferred embodiments, however, the adapter plate 112 is active, in that it includes the ability to receive electrical power from the handle and/or receive light from the handle. In the case of electrical power, electrical connections are provided to receive AC or DC power from powered laryngoscope handles and transmit it to base 114. According to some embodiments, the adapter plate 112 is active, and includes optical connections to receive optical power from certain optical powered laryngoscope handles (e.g. having its own light source) and transmit the optical power to base 114.

According to some embodiments, base 114 includes a physical handle and/or thumb ring which is designed, textured and dimensioned to serve as a convenient location for user to hold on to the assembly and rotate and manipulate light wand. According to some embodiments the thumb rest can be located on the adaptor plate, or elsewhere to facilitate easy grasping and manipulation by the user.

According to some embodiments, the base 114 includes a physical handle and/or thumb ring (or thumb rest), with electrical circuitry, which is adapted to receive electrical power from the adaptor 112, and to transmit the switched power to conducting wires along the stylet 116.

According to some embodiments, the base 114 includes a physical handle and/or thumb ring, with electrical circuitry and switching capability; which is adapted to receive electrical power from the adaptor 112, and to transmit the switched power to conducting wires along the stylet 116.

According to some embodiments, the base 114 includes a physical handle and/or thumb ring, with electrical circuitry, switching and a light source. The base 114 receives electrical power from adaptor to power light source.

According to some embodiments the based 114 includes a physical handle and/or thumb ring, with batteries, electrical circuitry and switching. The power can be switched and transmitted it along wires up the stylet 116.

According to some embodiments, the base 114 includes a physical handle and/or thumb ring, with batteries, electrical circuitry, switching and a light source. The batteries can be used to power light source, in which case the adapter 112 is passive and provides a physical connection only. According to some embodiments, the batteries in base 114 are used to supplement power from a standard laryngoscope handle so as to enable a higher level of brightness.

According to some embodiments, the base 114 includes a physical handle and/or thumb ring, with an optical light pipe, such as optical fiber(s), to receive light from base 114, and transmit the light to the stylet 116.

According to some embodiments, the electrical circuitry within the base 114 is adapted to control the electrical power in a manner that causes light source (in base or at lighted tip) to vary in certain patterns (flashes, dims/brightens, and/or colors).

According to some embodiments, stylet 116 is a flexible, hollow tube, with malleable mandrel insert. The hollow tube material is preferably a smooth non-sticking material such as Teflon that allows the lightwand to be easily inserted into the endotracheal tube without the need for the use of lubrication. The mandrel insert is preferable made of steel of an appropriate gauge so as to allow bending by the user while maintaining the bent shape while the endotracheal tube is being inserted through the patient's airway.

According to some embodiments, stylet 116 is a flexible, hollow tube, with malleable mandrel insert, with wires receiving electrical power from base 114 and transmitting it to the lighted tip 118. FIG. 1B shows a cross section of a stylet, according to some embodiments. Stylet tube wall 120 is preferably made of Teflon. Also shown is malleable rod/mandrel insert 122, and two insulated conductors 124 and 126.

According to some embodiments, stylet 116 is a flexible, hollow tube, with malleable mandrel insert, and a fiber optic light guide, such as optical fiber(s), designed to receive light from base 114, and transmit the light to lighted tip 118. FIG. 1C shows a cross section of a stylet, according to some embodiments. Stylet tube wall 120 is preferably made of Teflon. Also shown are malleable rod/mandrel insert 122, and an optical fiber bundle 134. According to some embodiments, the lightwand 110 is designed so as to mate with an ISO standard fiber-optic illuminated handle such as described in the publication 7376:2003(E) which is incorporated by reference herein.

According to some embodiments, lighted tip assembly 118 includes one or more incandescent or LED bulb(s), waterproof encapsulated, with various spatial orientations, radiating light with a certain pattern when receiving electrical power from stylet 116. According to some embodiments, the lighted tip 118 is designed to emit light at a 30-60 degree field of view aligned with the central longitudinal axis of the stylet 116.

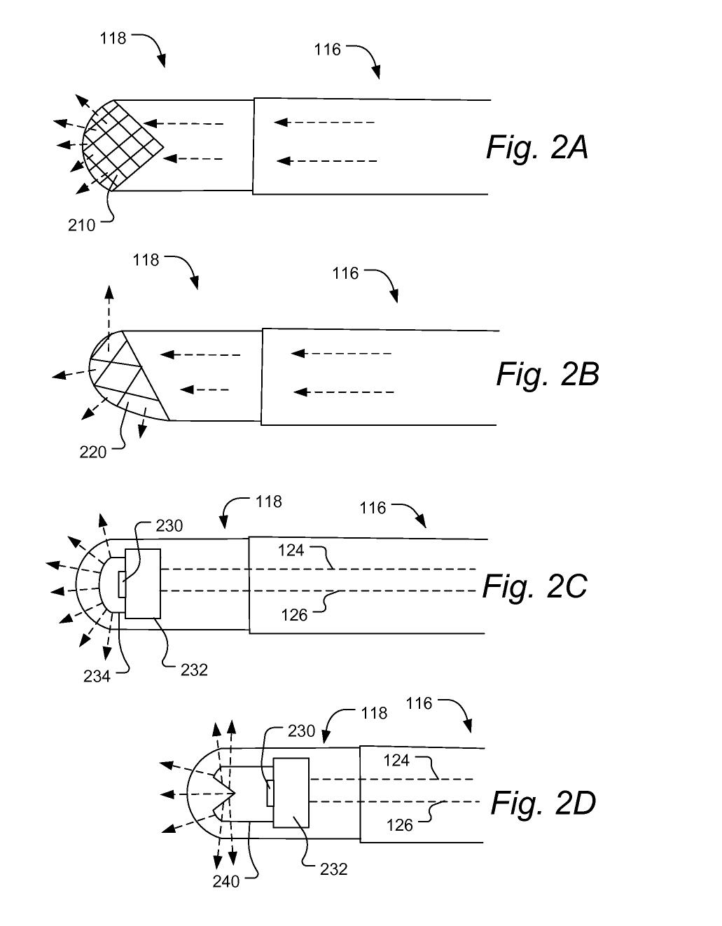

FIGS. 2A-2B illustrate a lighted tip assembly having multi-facetted lens according to some embodiments. In FIGS. 2A and 2B, lighted tip assembly 118 includes an optical lens assembly 210 and 220 respectively, each having one or more facets, radiating light with a certain pattern when receiving light from stylet 116. The light paths are shown in the broken-line arrows.

FIG. 2C illustrates a lighted tip assembly for a light wand according to some embodiments. The lighted tip assembly 118 is shown in this case with an LED 230 mounted on a board 232. According to some embodiments, LED 230 is in a bare form and is not mounted to a board. The light from LED 230 is controlled by the shape of the lens 234. According to some embodiments, the lens 234 is shaped to provide a 30-60 degree field of view aligned with the central longitudinal axis of the sylet 116. However, according to some embodiments the shape of lens 234 can be designed such that light is emitted along an equatorial 180 degree field of view aligned with the central longitudinal axis of the stylet 116. Light emitted at a 180 degree field of view can be used to aid a user during an airway intubation procedure on a patient by giving the user an indication of the location of the distal end of the device. According to some embodiments the light emitted from LED 230 includes infrared frequencies such that an infrared viewer can be used to facilitate intubation.

FIG. 2D illustrates a lighted tip assembly for a light wand according to some embodiments. The lighted tip assembly 118 is shown in this case with an LED 230 mounted on a board 232 (or LED 230 can be bare without being mounted to a board). In this case the lens 240 is designed to provide a split pattern of light with one portion of light directed forward at 30-60 degrees field of view and another portion of light directed at 170-190 degrees field of view. The split pattern can be useful in providing the user with both a direct indication of the location of the lighted tip from the 170-190 degree portion, as well as a distinctive cone-shaped pattern seen through the anterior neck tissues from the 30-60 degree portion.

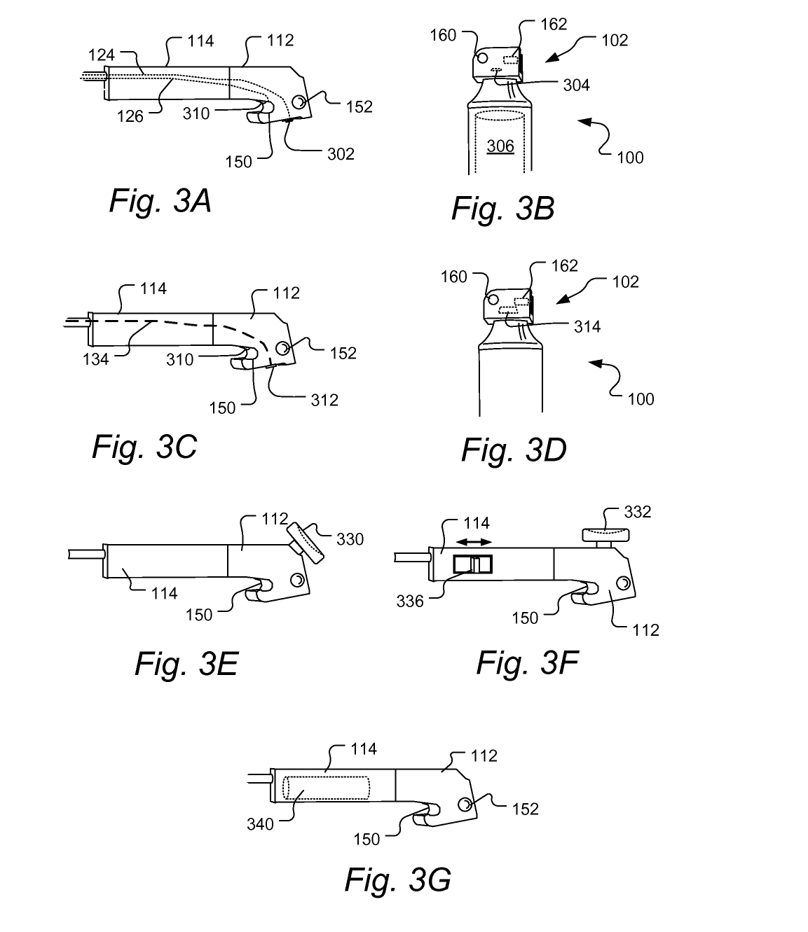

FIGS. 3A-3G illustrate further details of certain aspects of a light wand device that is mountable to a standard laryngoscope handle, according to some embodiments. FIGS. 3A and 3B show details of an adapter and anchor block, respectively, that include electrical connections for power supply by the handle to the light wand, according to some embodiments. The adapter plate 112 in this case has an electrical contact 302 that makes electrical contact with an electrical contact 304 on the anchor block 103 of laryngoscope handle 100, which in this case includes batteries such as battery 306. The contact 302 is electrically coupled to the conductive wire 124 running down the length of the stylet. The other conductor 126 is connected to part of the adapter plate 112 which makes electrical contact with the body of anchor block 102 of handle 100. Also shown in FIG. 3A is a retainer 310 to provide a more secure attachment mounting between the adapter plate 112 and anchor block 102.

FIGS. 3C and 3D show details of an adapter and anchor block, respectively, that include an illumination source in the handle and an optical guide (e.g. fiber optic) in the light wand, according to some embodiments. An optical receiver unit 312 is mounted on the bottom of adapter plate 112 that is positioned to receive light emitted by a light source in handle 100 (for example and emitter 314), and transmit the light to the fiber optic conductor 134 running down the length of the stylet.

FIG. 3E shows details of an adapter plate having a thumb rest, according to some embodiments. In this case, a thumb rest 330 is mounted at an angle as shown on adapter plate 112, and is textured and dimensioned to serve as a convenient location for user to hold on to the assembly and rotate and manipulate light wand during use.

FIG. 3F shows details of an adapter plate having a thumb rest and/or a switch, according to some other embodiments. In this case, a thumb rest 330 is mounted on the top of adapter plate 112 as shown. Additionally, in this case a sliding switch 336 is provided that can be used to turn on and off the light source in cases where electrical power is supplied by the handle (such as shown in FIGS. 3A and 3B) and/or where electrical power is supplied by batteries housed in the light wand base (such as shown in FIG. 3G). According to some embodiments, the switch is mounted in a different location and/or is of a different type. According to some embodiments, the switch is integrated into the thumb rest.

FIG. 3G illustrates a light wand base that includes a battery, according to some embodiments. Note that the battery 340 can be used to power the light source (such LEDs as shown in FIGS. 2C and 2D) alone, or can be used to boost or supplement electric power supplied by the handle (such as shown in FIGS. 3A and 3B).

According to some embodiments, system accessories may include a flexible light shield that is dimensioned so as to provide a temporary shield over external face, neck and throat area, to assist in the use of lightwand.

According to some embodiments, the entire system is designed to be single use, and according to other embodiments the system is designed to reused a number of times.

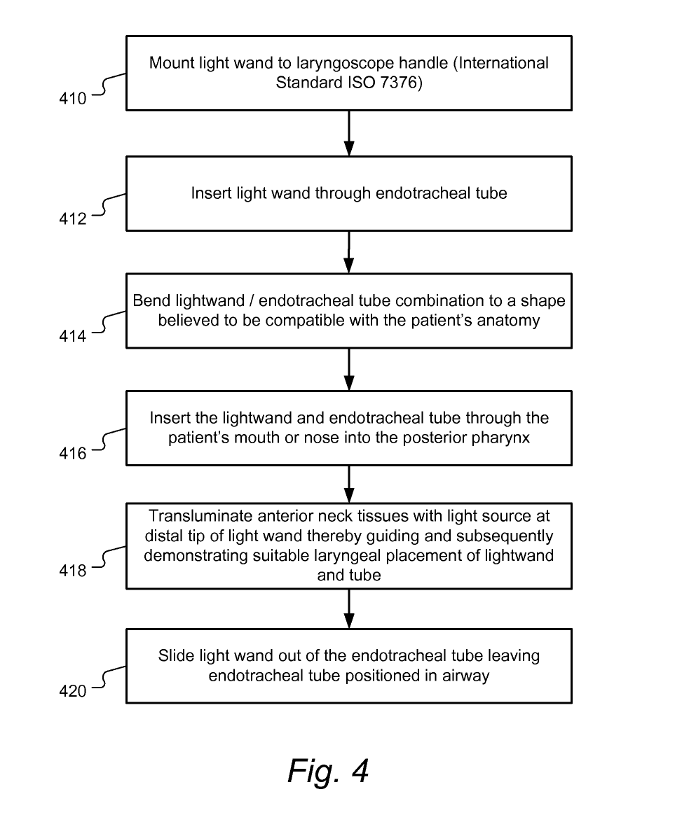

FIG. 4 is a flow chart showing some aspects of a method of intubating an airway of a patient, according to some embodiments. In step 410, the light wand is mounted to laryngoscope handle, such as one that conforms to International Standard ISO 7376). In step 412, the light wand is inserted through an appropriately dimensioned endotracheal tube. In step 414, the light wand and endotracheal tube combination is bent to a shape believed to be compatible with the patient's anatomy. In step 416, the light wand and endotracheal tube are inserted together through the patient's airway (e.g. orally or nasally). In step 418, the light source at distal tip of light wand is used to transilluminate the patient's anterior neck tissues, thereby demonstrating to the user when a suitable laryngeal or tracheal placement of lightwand and tube has been made. Following suitable placement, in step 410, the light wand is slid out of the endotracheal tube leaving the endotracheal tube positioned in airway.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the inventive body of work is not to be limited to the details given herein.

What is claimed is:

1. A device for use in airway intubation comprising:
a base portion including an adapter portion adapted for physical mounting to a laryngoscope handle; and
a flexible elongated member having a distal end and a proximal end, the proximal being fixed to the base portion, and the distal end including a light emitting tip portion capable of emitting light for facilitating airway intubation of a patient when the device is mounted to a laryngoscope handle.

2. A device according to claim 1 wherein the portion adapted for physical mounting to a laryngoscope handle is adapted for mounting to one or more laryngoscope handles conforming to International Standard ISO 7376.

3. A device according to claim 1 wherein the portion adapted for physical mounting to a laryngoscope handle is further adapted for electrical power connection with a laryngoscope handle.

4. A device according to claim 3 wherein the portion adapted for electrical power connection with a laryngoscope handle is adapted to receive electric power supplied by the laryngoscope handle for use in generating light for the light emitting tip portion.

5. A device according to claim 4 further comprising a light source powered at least in part by the electric power supplied by the laryngoscope handle.

6. A device according to claim 5 further comprising a switching unit operable by a user to turn on and off light generated by the light source.

7. A device according to claim 1 further comprising:
a light guide adapted and positioned to guide light received from a laryngoscope handle to the light emitting tip portion; and
a light receiver included in the portion adapted for physical mounting to a laryngoscope handle, the light receiver coupled to the light guide and adapted to receive light supplied by the laryngoscope handle and pass the received light through the light guide.

8. A device according to claim 1 wherein the base portion includes one or more batteries for supplying power for use in generating light for the light emitting tip portion.

9. A device according to claim 8 further comprising:
a light source for generating light for the light emitting tip portion, the light source being powered at least in part by the one or more batteries; and
a switching unit operable by a user to turn on and off light generated by the light source.

10. A device according to claim 9, wherein the portion adapted for physical mounting to a laryngoscope handle is further adapted to receive DC electric power supplied by the laryngoscope handle for use in generating light for the light emitting tip portion, the one or more batteries being used to supplement DC electric power supplied by the laryngoscope handle.

11. A device according to claim 1 wherein the base portion further includes a thumb rest portion positioned and dimensioned so as to facilitate grasping and manipulation of the device by a user during an air intubation procedure on a patient.

12. A device according to claim 1 wherein the device is adapted for oral intubation of a patient.

13. A device according to claim 1 wherein the device is adapted for nasal intubation of a patient.

14. A device according to claim 1 wherein the light emitting tip portion is adapted to emit light primarily in a 30 to 60 degree field of view aligned with a longitudinal axis of the distal end of the flexible elongated member.

15. A device according to claim 1 wherein the light emitting tip portion is adapted to emit light in directions perpendicular to a longitudinal axis of the distal end of the flexible elongated member, so as to aid a user during an airway intubation procedure on a patient by giving the user an indication of the location of the distal end of the device.

16. A device according to claim 1 wherein the light emitting portion is adapted to emit light from a plurality of locations near the distal end of the flexible elongated member.

17. A method of intubating a patient's trachea, the method comprising:
mounting a device according to claim 1 to a laryngoscope handle;
inserting the elongated member through an endotracheal tube; and
intubating the trachea of the patient by inserting the elongated member and endotracheal tube through the patient's airway, the intubation being facilitated by light from the light emitting distal tip portion transilluminating anterior neck tissues thereby demonstrating laryngeal or tracheal placement.

18. A method of intubating patient's trachea, the method comprising:
mounting a device to a laryngoscope handle, the device including a flexible elongated member having a light emitting distal tip portion, and an adapter portion adapted for physical mounting to the laryngoscope handle;
inserting the elongated member through an endotracheal tube; and
intubating the trachea of the patient by inserting the elongated member and endotracheal tube through the patient's airway.

19. A method according to claim 18 wherein the intubation is facilitated by light from the light emitting distal tip portion transilluminating anterior neck tissues thereby demonstrating laryngeal or tracheal placement.

20. A method according to claim 18 further comprising bending the elongated member and endotracheal tube to a shape believed to be compatible with the patient's anatomy prior to the insertion.

21. A method according to claim 18 wherein the laryngoscope handle conforms to International Standard ISO 7376.

22. A method according to claim 18 wherein the adapter portion adapted for physical mounting to a laryngoscope handle is further adapted for receiving DC electric power supplied by the laryngoscope handle for use in generating light for the light emitting tip portion.
